SEQUENCE LISTING (10 sequences)

SEQ ID NO: 1 - DNA, 704 bp, Homo sapiens
  CDS (102)...(449)
gcagttactgagaactcataagacgaagctaaaatccctcttcggatccacagtcaaccgccctgaacacatcctgcaaaaagcccagagaaaggagcgccatggattactacagaaaatatgcagctatctttctggtcacattgtcggtgtttctgcatgttctccattccgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatcttaaatgttttaccaagtgctgtcttgatgactgctgattttctggaatggaaaattaagttgtttagtgtttatggctttgtgagataaaactctccttttccttaccataccactttgacacgcttcaaggatatactgcagctttactgccttcctccttatcctacagtacaatcagcagtctagttcttttcatttggaatgaatacagcattaagcttgttccactgcaaataaagccttttaaatcatc

SEQ ID NO: 2 - protein, 116 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 3 - DNA, 879 bp, Homo sapiens
  CDS (366)...(860)
tccagcacctttctcgggtcacggcctcctcctggctcccaggaccccaccataggcagaggcaggccttcctacaccctactccctgtgcctccagcctcgactagtccctagcactcgacgactgagtctctgaggtcacttcaccgtggtctccgcctcacccttggcgctggaccagtgagaggagagggctggggcgctccgctgagccactcctgcgcccccctggccttgtctacctcttgccccccgaggggttagtgtcgagctcaccccagcatcctatcacctcctggtggccttgccgcccccacaaccccgaggtataaagccaggtacacgaggcaggggacgcaccaaggatggagatgttccaggggctgctgctgttgctgctgctgagcatgggcgggacatgggcatccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagcccatcccgactcccggggccctcggacaccccgatcctcccacaataaaggcttctcaatccgc

SEQ ID NO: 4 - protein, 165 aa, Homo sapiens
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 5 - DNA, 22 bp, Artificial Sequence
  primer
gaatgacctgttctttgaagtt

SEQ ID NO: 6 - DNA, 22 bp, Artificial Sequence
  primer
ttttgttgttcatctcggagcc

SEQ ID NO: 7 - DNA, 24 bp, Artificial Sequence
  primer
ccttccgaagtttctggcagcagc

SEQ ID NO: 8 - DNA, 25 bp, Artificial Sequence
  primer
ggctgtcagagcctcgtggctttgg

SEQ ID NO: 9 - DNA, 23 bp, Artificial Sequence
  primer
gtaatgatcagtcaacgggggac

SEQ ID NO: 10 - DNA, 24 bp, Artificial Sequence
  primer
ccagcaagcttgcaaccttaacca

(19) United States
(12) Patent Application Publication
(10) Pub. No.: US 2006/0063713 A1
(43) Pub. Date: Mar. 23, 2006

(54) Methods for restoring immune balance for the treatment of T-cell mediated diseases

(75) Inventor: Ji-Won Yoon, Vernon Hills, IL (US)

Correspondence Address: MCDERMOTT, WILL & EMERY, 4370 LA JOLLA VILLAGE DRIVE, SUITE 700, SAN DIEGO, CA, 92122, US

(73) Assignee: Biotech Institute for international Innovation, Inc.

(21) Appl. No.: 11/219,339

(22) Filed: Sep. 1, 2005

Related U.S. Application Data
(60) Provisional application No. 60/606,831, filed on Sep. 2, 2004.

(51) Int. Cl. A61K 38/22

(52) U.S. Cl. 514/12

(57) ABSTRACT

The invention provides a method of treating a T cell mediated disease. The method includes administering an effective amount of hCG to an asymptomatic subject over a period of time sufficient to restore persistent immune balance between regulatory and effector T cells, wherein restoring the persistent immune balance results in a reduction in the severity of the T cell mediated disease. The invention also provides a method of preventing T cell mediated disease in a pre-diseased subject. The method includes administering an effective amount of hCG to a subject at risk of developing a T cell mediated disease for sufficient duration to confer persistent immune balance between regulatory and effector T cells functions. Also provided is a pharmaceutical composition having an effective amount of substantially purified hCG in a pharmaceutically acceptable medium and a formulations thereof suitable for administration to a subject.

This application is based on, and claims the benefit of, U.S. Provisional Application No. 60/606,831, filed Sep. 2, 2004, entitled Methods for Restoring Immune Balance for the Treatment of T-Cell Mediated Diseases, and is incorporated herein by reference.

### BACKGROUND OF THE INVENTION

This invention relates generally to the therapeutic treatment of diseases and, more specifically to the modulation of immune system regulation for the treatment of autoimmune diseases.

Higher organisms are characterized by an immune system which protects them against invasion by potentially deleterious substances or microorganisms. When a substance, termed an antigen, enters the body, and is recognized as foreign, the immune system mounts both an antibody-mediated response and a cell-mediated response. Cells of the immune system termed B lymphocytes, or B cells, produce antibodies that specifically recognize and bind to the foreign substance. Other lymphocytes termed T lymphocytes, or T cells, both effect and regulate the cell-mediated response resulting eventually in the elimination of the antigen.

A variety of T cells are involved in the cell-mediated response. Some induce particular B cell clones to proliferate and produce antibodies specific for the antigen. Others recognize and destroy cells presenting foreign antigens on their surfaces. Certain T cells regulate the response by either stimulating or suppressing other cells.

While the normal immune system is closely regulated, aberrations in immune response are not uncommon. In some instances, the immune system functions inappropriately and reacts to a component of the host as if it were, in fact, foreign. Such a response results in an autoimmune disease, in which the host's immune system attacks the host's own tissue. T cells, as the primary regulators of the immune system, directly or indirectly effect such autoimmune pathologies.

Numerous diseases are believed to result from autoimmune mechanisms. Prominent among these are rheumatoid arthritis, systemic lupus erythematosus, multiple sclerosis, Type I diabetes, myasthenia gravis and pemphigus vulgaris. Autoimmune diseases affect millions of individuals world-wide and the cost of these diseases, in terms of actual treatment expenditures and lost productivity, is measured in billions of dollars annually. At present, there are no known effective treatments for such autoimmune pathologies. Usually, only the symptoms can be treated, while the disease continues to progress, often resulting in severe debilitation or death.

Thus, there exists a need for an effective means of curing or ameliorating T cell mediated pathologies. Such a treatment should ideally control the inappropriate T cell response, rather than merely reducing the symptoms. The present invention satisfies this need and provides related advantages as well.

### SUMMARY OF THE INVENTION

The invention provides a method of treating a T cell mediated disease. The method includes administering an effective amount of hCG to an asymptomatic subject over a period of time sufficient to restore persistent immune balance between regulatory and effector T cells, wherein restoring the persistent immune balance results in a reduction in the severity of the T cell mediated disease. The invention also provides a method of preventing T cell mediated disease in a pre-diseased subject. The method includes administering an effective amount of hCG to a subject at risk of developing a T cell mediated disease for sufficient duration to confer persistent immune balance between regulatory and effector T cells functions. Also provided is a pharmaceutical composition having an effective amount of substantially purified hCG in a pharmaceutically acceptable medium and a formulations thereof suitable for administration to a subject.

### BRIEF DESCRIPTION OF THE DRAWINGS

FIG. 1 shows that hCG treatment prevents the development of diabetes in female NOD mice.

FIG. 2 shows hCG treatment prevents the adoptive transfer of diabetes to NOD.SCID mice by increasing the CD4⁺CD25⁺ regulatory T cell population.

FIG. 3 shows hCG treatment decreases the number and proliferation of CD8⁺ T cells.

FIG. 4 shows that hCG treatment decreases the production of Th1 cytokines.

FIG. 5 shows hCG treatment inhibits macrophage activation.

FIG. 6 shows the nucleotide and deduced amino sequence for the hCG α chain (SEQ ID NOS:1 and 2, respectively).

FIG. 7 shows the nucleotide and deduced amino sequence for the hCG β chain (SEQ ID NOS:3 and 4, respectively).

### DETAILED DESCRIPTION OF THE INVENTION

The invention is directed to methods of preventing or reducing the severity of T cell mediated diseases. The methods of the invention relate to the discovery that treatment of a subject with human chorionic gonadotropin (hCG) results in the prevention of type I diabetes and induces the up-regulation of CD4⁺CD25⁺ regulatory T cells while down-regulating effector T cells and their cytokines, such as those involved in a Th1 response. This discovery extends the biological role of hCG and allows its use in the treatment of a variety of diseases characterized by immune deviation and/or abnormal cytokine release.

The various embodiments of the methods of the invention derivate from the results described below in the Examples. Briefly, these studies indicate that hCG treatment prevents the onset of autoimmune diabetes in subjects lacking overt clinical symptoms. In subjects exhibiting clinical symptoms of diabetes, hCG treatment is able to reduce the severity or progression of the disease. Treatment also resulted in selectively increasing regulatory CD4⁺CD25⁺ T cell populations while depletion of these T cells prevented adoptive transfer of the hCG mediated regulatory effect observed in treated animals. Complete loss of this preventative effect in populations depleted of CD4⁺CD25 regulatory T cells indicates that CD4⁺CD25⁺ T cells are responsible for the prevention of autoimmune diabetes in hCG-treated subjects.

Further, treatment with hCG additionally decreased CD8⁺ T cells, but not CD4⁺ effector T cells and also suppressed Th1 immune responses, indicating that the decrease in both cytotoxic CD8⁺ T cells and the Th1 immune response contributes to the prevention of diabetes in hCG-treated subjects. Other studies described in the Examples below indicate that hCG-induced inhibition of macrophage function also can contribute to the prevention of diabetes. Taken together these results further indicate that hCG is an important down-regulator of immune effector cell function. Recombinant hCG also was show to have the same effect on immune regulation as purified hCG, indicating that hCG itself, rather than some contaminant in purified hCG preparations, is responsible for the autoimmune preventive effect of hCG.

As used herein, the term “hCG” is intended to mean a heterodimeric hCG protein consisting of an a chain polypeptide of about 200 amino acids and α chain polypeptide consisting of about 150 amino acids. The amino acid sequence of hCG can be found described in, for example, Morgan et al. J. Biol. Chem. 250:5247-58 (1975) (SEQ ID NOS:2 (α chain) and 4 (β chain)). The nucleotide sequences of hCG can be found described in, for example, Fiddes and Goodman, Nature 281:351-6 (1979) (SEQ ID NO:1 (α chain)) and Fiddes and Goodman, Nature 286:684-87 (1980) (SEQ ID NO:3 (β chain)). Heterodimeric hCG also includes homologs of hCG as well as orthologs and nonorthologous displacements from species other than human which have substantially the same amino acid sequence, or encoded by substantially the same nucleotide sequence as the hCG described above, and which has substantially the same immunomodulatory function as hCG described herein. Heterogeneous preparations of hCG also are included within the meaning of the term so long as such preparations contain an enriched amount of heterodimeric hCG. A specific example of a heterogeneous preparation being enriched in heterodimeric hCG is described below in the Examples. The term “hCG” as it is used herein is intended to exclude the 400-2000 Dalton fraction of c-hCG or u-hCG, and the 17 amino acid peptide termed NMPF peptide, as described by Khan et al., Human Immunol. 62:1315-23 (2001). Similarly, the term “hCG” as it is used herein also is intended to exclude short peptides of about 3-20 amino acids derived from the β subunit of hCG, including the NMPF peptide, as described in WO 01/072831A2.

The term hCG is intended to include polypeptides having substantially the same amino acid sequence compared to the sequences shown as SEQ ID NOS:1 and 3. Therefore, substantially the same hCG amino acid sequence refers the described hCG amino acid sequences or other sequences having minor additions, deletions or substitutions that do not substantially effect the ability of the sequence to regulate T cells and restore immune balance as described herein. Similarly, a fragment, portion or segment of the described hCG α sequence, β sequence or the αβ heterodimer can be used so long as it is sufficiently characteristic of hCG or a fragment thereof to cause an effective immune response that regulates desired T cell populations. Such variations in the sequence can easily be made, for example, by synthesizing an alternative sequence. The alternate sequence can then be tested, for example by administration to a vertebrate, to determine its effectiveness. Similarly, a nucleic acid encoding hCG is intended to include nucleic acids having substantially the same nucleotide sequence compared to the sequences shown as SEQ ID NOS:2 and 4.

Immunoregulatory fragments of hCG refer to a portion of the sequences shown as SEQ ID NOS:1 and 3 that retain immunoregulatory function substantially the same or greater than that exhibited by hCG. Similarly, immunoregulatory fragments of hCG includes hCG fragments in conjunction with or combined with additional sequences or moieties so long as such hCG fragments maintain their ability to regulate T cell populations and restore immune balance. An hCG fragment also includes minor modifications in sequence, as described above for substantially the same hCG amino acid sequence. Immunoregulatory hCG fragments exclude the 400-2000 Dalton fraction of c-hCG or u-hCG, and the 17 amino acid peptide termed NMPF peptide, as described by Khan et al., Human Immunol. 62:1315-23 (2001). Similarly, the term “hCG” as it is used herein also is intended to exclude short peptides of about 3-20 amino acids derived from the β subunit of hCG, including the NMPF peptide, as described in WO 01/072831A2.

As used herein, the term “T cell mediated disease” is intended to mean a condition in which an inappropriate T cell response is a component of the pathology. The term is intended to include both diseases directly mediated by T cells and those diseases in which an inappropriate T cell response contributes to damage resulting from the production of autoimmune antibodies. The term is intended to encompass both T cell mediated autoimmune diseases and unregulated clonal T cell replication. Therefore, a T cell mediated disease includes T cell mediated conditions exhibiting clinically recognizable symptoms as well as T cell mediated dysfunctions. Specific examples of T cell mediated diseases include type 1 diabetes, insulitis, Graves' disease, rheumatoid arthritis (RA), multiple sclerosis (MS), systemic lupus erythematosus, myasthenia gravis, pemphigus vulgaris, Hashimoto's Thyroditis, Autoimmune Uveitis, Sjogren's syndrome, Dermamyositis and Addison's disease.

As used herein, the term “effective amount” is intended to mean an amount of hCG which is effective to elicit a change in the immune balance by up-regulation of regulatory T cells and down-regulation of effector T cell function to reduce the severity, prolong progression or inhibit the occurrence of a T cell mediated disease. The dosage of hCG sufficient to achieve such a restoration of immune balance will depend, for example, on the T cell mediated disease to be treated, the route and form of administration, the concentration of the preparation being administered, the weight and condition of the subject, and previous or concurrent therapies. Exemplary dosages considered to be an effective amount for treatment of type 1 diabetes are described further below. Dosages considered to be an effective amount for other T cell mediated diseases can be determined by those skilled in the art using the teachings and guidance provided herein. For example, the amount can be extrapolated from in vitro assays or in vivo models that are predictive of human T cell mediated diseases. One skilled in the art will recognize that the condition of the patient can be monitored throughout the course of therapy and that the amount of hCG, or a formulation thereof, can be adjusted according to well known diagnostic parameters. Exemplary effective amounts for the treatment of a human subject include hCG amounts ranging from about 5 IU/kg body weight to about 500 IU/kg body weight, particularly from about 10 IU/kg body weight to about 250 IU/kg body weigh and more particularly from about 15 IU/kg body weight to about 200 IU/kg body weight. Effective amounts for the treatment of a human subject also includes all values in between these exemplary ranges.

As used herein, the term “subject” means any vertebrate, including humans, capable of having a T cell mediated disease. A subject includes, for example, vertebrates that are symptomatic, asymptomatic, exhibiting T cell mediated dysfunction, at risk of developing a T cell mediated disease or suspected of having a T cell mediated disease.

By “substantially pure,” it is meant that the hCG is substantially free of other biochemical moieties with which it is normally associated in nature. Such substantially pure hCG or fragments thereof, for instance, can be synthesized, produced recombinantly by means known to those skilled in the art. In addition, whole hCG chains or heterodimers can be enzymatically treated to produce such fragments.

Human chorionic gonadotropin (hCG) is a heterodimeric placental glycoprotein (Pierce, et al. Ann. Rev. Biochem 50:465-495 (1981)) required to maintain pregnancy (Healy, Gynecologic Endocrinology 4th Ed, Plenum Publishing Co New York, 23-52 (1987); Ascoli, et al. Endocr. Rev. 23:141-174 (2002)). The level of hCG doubles every 2 days between 1 and 9 wk of pregnancy and then decreases to 10% of the peak value during the second and third trimester (Healy supra). The symptoms of autoimmune diseases such as rheumatoid arthritis, Crohn's disease, and multiple sclerosis are attenuated during pregnancy (Buyon, et al Clin. Immunol. Immunopathol. 78:99-104 (1996); Wilder, Ann. N.Y. Acad. Sci. 840:45-50 (1998); Da Silva, et al. Clin. Rheumatol. 11:189-194 (1992); Castiglione, et al. Ital. J. Gastroenterol 28:199-204 (1996); Confavreux, et al. N. Engl. J. Med. 339:285-291 (1998)). In pregnant women, the expression of Th1 cytokines such as IL-2 and IFN-γ is significantly decreased, and the expression of IL-18 mRNA, an inducer of IFN-γ in T lymphocytes and NK cells, is inversely correlated with serum levels of hCG in pregnant women (Kruse, et al. Clin. Exp. Immunol. 119:317-322 (2000)). Although hCG levels appear to correlate with an immunoregulatory role in addition to its reproductive role such as the maintenance of pregnancy, there are many other changes occurring during these stages of development which could account for these observations.

At least one report has theorized that hCG functions as an immunoregulator, but found that within a preparation of clinical grade hCG (c-hCG) immunoregulatory activity co-purified with a low molecular weight peptide fraction. In particular, a specific low molecular weight fraction ranging from 400-2000 Daltons of hCG contained a factor that appeared to reduce diabetes in mice (Khan, et al. Human Immunol. 52:1315-1323 (2001)). A synthetic six amino acid peptide having the sequence VLPALP was suggested to have the same activity as the low molecular weight hCG fraction. Moreover, intact hCG and hCG subunits were specifically ruled out as active immunoregulatory factors. Fractions greater than 2,000 Daltons of c-hCG or hCG isolated from first trimester pregnancy urine (u-hCG) also failed to show an inhibitory effect on diabetes development. Similarly, recombinant hCG (r-hCG), highly purified urinary hCG, r-α-hCG subunit or r-β-hCG subunit or preparations from Profasi and APL also failed to show an inhibitory effect on diabetes development.

The invention provides a method of treating a T cell mediated disease. The method includes restoring immune balance in a subject by administering an effective amount of heterodimeric hCG to an asymptomatic subject over a period of time sufficient to confer persistent changes and restoration of the balance between regulatory and effector T cells. Treatment using the methods of the invention includes the prevention of disease onset in a subject when administered prior to or concurrent with the occurrence of autoimmune dysfunction. Treatment using the methods of the invention also includes reducing the progression or symptoms of the disease in a subject when administered subsequent to the occurrence of autoimmune dysfunction, but prior to or after clinical manifestations.

The methods of the invention are applicable to a wide variety of autoimmune and/or aberrant proliferative T cell mediated diseases. Exemplary T cell mediated diseases that can be treated using the method of the invention include type 1 diabetes, insulitis, Graves' disease, rheumatoid arthritis, multiple sclerosis, systemic lupus erythematosus, myasthenia gravis and pemphigus vulgaris. In specific instances, the invention is described below with reference to the treatment of type 1 diabetes as an exemplary T cell mediated disease. However, given the teachings and guidance provided herein, those skilled in the art will understand that the methods of the invention are equally applicable to the T cell mediated diseases enumerated above as well as other such autoimmune and/or aberrant proliferative T cell mediated diseases known in the art.

Animal models have contributed significantly to the understanding of the immunological mechanisms of T cell mediated diseases, including autoimmune disease. Exemplified herein is the treatment of the nonobese diabetic (NOD) mouse, which is a widely used and credible animal model for the study of human autoimmune type 1 diabetes. Type 1 diabetes results from the destruction of pancreatic β cells by T cell-mediated autoimmune responses (Notkins, J. Biol. Chem. 277:43545-43548 (2002); Bach, et al. Annu. Rev. Immunol. 19:131-161 (2001); Tisch, et al. Cell 85:291-297 (1996); Yoon, et al. Encyclopedia of Immunology 2d, Academic Press, London 1390-1398 (1998)). These autoimmune responses can result from a breakdown of immune balance, including a decrease in regulatory T cells and an increase in autoreactive effector T cells and their cytokines (Kukreja, et al. J. Clin. Invest. 109:131-140 (2002)).

Analysis hCG treatment in the NOD model showed that treatment of young NOD mice with hCG (50 IU/mouse) completely prevented autoimmune diabetes. Splenic T cells from hCG-treated NOD mice failed to induce diabetes in NOD.SCID mice. CD4⁺CD25⁺ regulatory T cells were significantly increased in the spleen and pancreatic LN of hCG-treated NOD mice. Depletion of these regulatory T cells in hCG-treated NOD mice abolished the preventive effect of hCG. In addition, hCG down-regulated both the Th1 immune response and CD8⁺ T cell proliferation, confirming that hCG can prevent autoimmune diabetes in NOD mice by restoring the immune balance by up-regulating regulatory T cells and down-regulating effector T cell function.

Another credible animal model for treatment of T cell mediated diseases is experimental allergic encephalomyelitis (EAE), which is an autoimmune disease of the central nervous system that can be induced in mice and rats by immunization with myelin basic protein (MBP). The disease is characterized clinically by paralysis and mild wasting and histologically by a perivascular mononuclear cell infiltration of the central nervous system parenchyma. The disease pathogenesis is mediated by T cells having specificity for MBP. Multiple clones of MBP-specific T cells have been isolated from animals suffering from EAE and have been propagated in continuous culture. After in vitro stimulation with MBP, these T cell clones rapidly induce EAE when adoptively transferred to healthy hosts. Given the teachings and guidance provided herein, an EAE can similarly be used as an animal model for confirming the effectiveness of hCG for the treatment of multiple sclerosis. Other credible animal models well known to those skilled in the art also can be used to confirm the effectiveness of hCG administration for the treatment of a wide variety of T cell mediated diseases.

The methods of the invention can be used to treat subjects that are symptomatic, asymptomatic, exhibiting T cell mediated dysfunction, at risk of developing a T cell mediated disease or suspected of having a T cell mediated disease. Prevention of a T cell mediated disease can be accomplished in asymptomatic individuals where administration of hCG occurs prior to or concurrent with the occurrence of autoimmune dysfunction or immune imbalance. Thus, prior to deregulation of regulatory T cells, effector T cell or both regulatory T cells and effector T cells, administration of an effective amount can prevent the onset of a T cell mediated disease.

Similarly, the methods of the invention include treatment of pre-T cell mediated disease subjects with hCG to restore immune balance and prevent the occurrence of a T cell mediated disease. Pre-T cell mediated disease subjects include, for example, subjects at risk of developing a T cell mediated disease. As with treatment for reducing the severity or progression of a T cell mediated disease, prevention of a T cell mediated disease can occur, for example, by up-regulating regulatory T cells, down-regulating effector T cells or both up-regulating regulatory T cells and down-regulating effector T cells.

Reduction in the progression or symptoms of a T cell mediated disease can be accomplished in asymptomatic individuals where administration of hCG occurs subsequent to the occurrence of autoimmune dysfunction. Following deregulation of regulatory T cells, effector T cell or both regulatory T cells and effector T cells, administration of an effective amount of hCG can slow or inhibit the further progression of a T cell mediated disease.

Therefore, increased efficacy of hCG treatment using the methods of the invention can correspond to the stage of the T cell mediated disease sought to be treated. For example, greater effectiveness can be achieved where treatment is initiated prior to the occurrence of overt clinical symptoms. However, reduction of symptoms or progression of the disease can occur where treatment initiates following clinical diagnosis. Prevention can be achieved, for example, where the subject is asymptomatic and before progression of autoimmune dysfunction or prophylactically to maintain immune function and balance.

Administration of hCG to a subject having a T cell mediated disease results in an immunomodulatory effect of T cells that restores immune balance between regulatory and effector T cells. In this regard, the proportions of regulatory and effector T cells or their immunological function is changed to or toward a non-diseased state. This modulation or change in the balance between regulatory and effector T cells prevents or reduces the progression of the T cell mediated disease. Similarly, administration of hCG to a subject at risk of having a T cell mediated disease results in an immunomodulatory effect of T cells that maintains immune balance between regulatory and effector T cells. Accordingly, the proportions of regulatory and effector T cells or their immunological function is refractory to change toward a diseases state in treated subjects and prevents occurrence of the disease.

hCG mediated immunomodulation conferring or restoring immune balance between regulatory and effector T cells includes the up-regulation of, for example, CD4⁺CD25⁺ regulatory T cells. hCG mediated immunomodulation conferring or restoring immune balance between regulatory and effector T cells also includes the down-regulation of, for example, CD8⁺ effector T cells as well as suppression of a Th1 immune response. Immunomodulation mediated by hCG additionally includes both the up-regulation of CD4⁺CD25⁺ regulatory T cells as well as down-regulation of CD8⁺ effector T cells and/or suppression of a Th1 immune response. This multifaceted immune regulation allows for effective treatment of a wide variety of T cell mediated diseases.

The methods of the invention employ heterodimeric hCG for the treatment of diabetes and other T cell mediated diseases. The anti-diabetic effect of hCG is optimal when the heterodimeric hCG protein, containing both the a chain and the β chain, is administered to a subject susceptible to type 1 diabetes or another T cell mediated disease. Heterodimeric hCG exhibits greater therapeutic efficacy than that which can be obtained using either hCG subunit alone or small peptide fragments derived, for example, from the β subunit of hCG. For example, the increased therapeutic efficacy which can be obtained using heterodimeric hCG includes increases of about 2-, 3-, 4-, 5-, 6-, 7-, 8-, 9- or 10-fold or greater compared to the hCG α subunit, the β subunit or a small peptide fragment thereof. Because heterodimeric hCG is more efficacious substantially lower doses can be administered for any given administration as well as over the course of treatment.

The course of hCG treatment can be adjusted depending on the stage of the disease. For example, in pre-disease stages dosages in humans can be extrapolated from mouse or other animal model dosages ranging from about 10-100 IU/mouse, preferably from about 25-75 IU/mouse, more preferably from about 40-60 IU/mouse. A particularly useful dosage is about 50 IU/mouse or comparable human equivalent. Dosages similar or identical to these also can be administered to subjects exhibiting autoimmune dysfunction, but prior to clinical manifestations. Such dosages can be administered in various combinations of amount and number of administrations to yield about 250 IU/mouse per week or comparable human equivalent. Individual dosages can be increased where diagnostic indicators suggest a short treatment period or a less frequent administration is beneficial. Alternatively, individual dosages can be decreased where longer durations of treatment or more frequent administrations are warranted. Formulations that allow for timed-release of hCG can provide for the continuous release of a smaller amount of hCG than would be administered as a single bolus dose. Exemplified below is the administration of 5 doses per week of 50 IU/mouse or comparable human equivalent. Frequent administrations provide the advantage of supplying a steady in vivo concentration of hCG over the course of treatment. Exemplary effective amounts for the treatment of a human subject include hCG amounts ranging from about 5 IU/kg body weight to about 500 IU/kg body weight, particularly from about 10 IU/kg body weight to about 250 IU/kg body weigh and more particularly from about 15 IU/kg body weight to about 200 IU/kg body weight. Effective amounts for the treatment of a human subject also includes all values in between these exemplary ranges.

Given the teachings and guidance provided herein, those skilled in the art will understand that various other combinations of hCG amount and administration frequency can be utilized in the method of the invention to achieve the same restoration of immune balance. Moreover, those skilled in the art can readily determine the effect of a particular treatment regime by administering a particular dosage to a predictable animal model and determining the effect on immune balance. Treatment regimes that up-regulate regulatory T cells and down-regulate effector T cell functions as described herein will constitute an effective amount of hCG in the methods of the invention. For example, the in vitro assays and in vivo models described below, as well as other methods known to those skilled in the art, can similarly be used to determine appropriate dosage regimes in regard to timing of administrations, number of administrations and amount per administration of hCG to treat or inhibit a T cell mediated disease. Similarly, the assays and models described above and below also can be routinely used to make and identify new, modified or improved hCG compositions or formulations. Similarly, the hCG proteins of the invention can be combined with various pharmaceutically acceptable mediums or carriers to determine an optimal or enhanced composition in which hCG can be administered or which may augment or stabilize the immunomodulatory activity of hCG.

In addition, hCG also can be administered in combination with other immunoregulators to augment the efficacy of the hCG treatment against diabetes or other T cell mediated diseases. Exemplary immunoregulators effective for augmenting the efficacy of hCG treatment include, for example, anti-CD3 antibody, Sirolimus, Tacrolimus, or Cyclosporin. Various other immunoregulators are well known to those skilled in the art and also can be used in conjunction with hCG treatment following the teachings and guidance described herein. Administration of such other immunoregulators can be, for example, simultaneously with hCG administration such as in the same admixture. Administration of such other immunoregulators also can occur, for example, concurrently with hCG such as during the same treatment setting but administered in a dose separate from hCG. Administration of such other immunoregulators also can occur, for example, temporally independent from administration of hCG such as before or after hCG administration or in between multiple hCG administrations. Dosages and timing of administration of such other immunoregulators are well known in the art. Given the teachings and guidance provided herein, those skilled in will know the amount and treatment regimen to use in combination with hCG administration to achieve a beneficial increase in the treatment of diabetes or other T cell mediated diseases.

The duration of treatment also can vary depending on the stage of the disease. For example, in pre-diseased subjects treatment can commence based on risk of exhibiting a T cell mediated disease. Accordingly, prophylactic applications are warranted in diseases where the autoimmune mechanisms precede the onset of overt clinical disease. Thus, subjects with familial history of disease and predicted to be at risk by reliable prognostic indicators can be treated prophylactically to interdict autoimmune mechanisms prior to their onset. Administration can proceed until susceptibility or risk of occurrence is reduced. Persistent immune balance of regulatory T cells and effector T cell functions is one indicator for terminating or attenuating treatment of a pre-diseased subject. The duration of such treatments can include, for example, days, weeks, months or years.

Dosages and effective amounts of hCG can differ for prophylactic treatments compared to diseased subjects. For example, in diseased subjects, the defense mechanism of immune system to autoreactive T cells can be almost completely destroyed. However, in the subjects for prophylactic treatment, the autoreactive T cells are developing and the defense mechanisms against these cells can be considerably retained. Therefore, if desired, an effective amount of hCG administered for prophylactic purposes can be lower, including significantly lower, compared an effective amount administered for the purposes of treating a subject exhibiting overt symptoms of diabetes or another T cell mediated disease. Effective amounts for treating a subject exhibiting overt symptoms has been described previously.

Similarly, a subject exhibiting autoimmune dysfunction mediated by aberrant T cells or definite clinical diagnosis of a particular autoimmune disease will warrant the administration of hCG or a formulation thereof. A specific example of an autoimmune dysfunction occurring prior to clinical manifestations is the occurrence of lymphocyte infiltration in pancreatic islet cells, or insulitis, prior to overt diabetic symptoms. Duration of administration can proceed until immune balance is restored, which can include, for example, days, weeks, months or years. Indicators of immune balance restoration include, for example, reduction in the progression or severity of the disease, regression of the autoimmune dysfunction or prevention of the occurrence of clinical symptoms. Dosages, effective amounts and treatment regimes for autoimmune dysfunctions include those described previously.

Administration of hCG, or a formulation thereof, can be delivered systemically, such as intravenously or intra-arterially. An hCG protein also can be administered locally at a site of the T cell mediated disease. Appropriate sites for administration of hCG are known or can be determined by those skilled in the art depending on the clinical indications of the subject being treated. For example, heterodimeric hCG having an immune balance restoration activity described above can be provided as isolated and substantially purified proteins or as a substantially purified protein in a pharmaceutically acceptable formulation using formulation methods well known to those skilled in the art. These formulations can be administered by standard routes, including for example, topical, transdermal, intraperitoneal, intracranial, intracerebroventricular, intracerebral, intravaginal, intrauterine, oral, rectal or parenteral (e.g., intravenous, intraspinal, subcutaneous or intramuscular) routes. In addition, hCG can be incorporated into biodegradable polymers allowing for sustained release of the compound, the polymers being implanted in the vicinity of where drug delivery is desired, for example, at the site of a T cell mediated disease or implanted so that the hCG is released systemically over time. Osmotic minipumps also can be used to provide controlled delivery of defined concentrations of hCG through cannulae to the site of interest, such as directly into pancreatic islet cells or into the vascular supply of such cells. Biodegradable polymers and their use are described, for example, in detail in Brem et al., J. Neurosurg. 74:441-446 (1991).

Therefore, the invention provides a method of preventing T cell mediated disease in a pre-diseased subject. The method comprises administering an effective amount of hCG to a subject at risk of developing a T cell mediated disease for sufficient duration to confer persistent changes onto regulatory and effector T cells functions. The T cell mediated disease can be type 1 diabetes. Persistent regulatory and effector T cell changes can include the up-regulation of regulatory T cell functions, the down-regulation of effector T cell functions or both the up-regulation of regulatory T cell functions and the down-regulation of effector T cell functions. The up-regulation of regulatory T-cell functions can include an increase in the CD4⁺CD25⁺ regulatory T-cell population. The down-regulation of effector T cell functions can include a selective inhibition of CD8⁺ T cell proliferation.

Also provided is a method of treating or reducing the severity a T cell mediated disease prior to overt clinical symptoms. The method comprises administering an effective amount of hCG to a subject exhibiting autoimmune dysfunction mediated by aberrant T cells for sufficient duration to confer persistent changes onto regulatory and effector T cells functions. The T cell mediated disease can be type 1 diabetes. An autoimmune dysfunction mediated by aberrant T cells can be insulitis. Persistent regulatory and effector T cell changes can include the up-regulation of regulatory T cell functions, the down-regulation of effector T cell functions or both the up-regulation of regulatory T cell functions and the down-regulation of effector T cell functions. The up-regulation of regulatory T-cell functions can include an increase in the CD4⁺CD25⁺ regulatory T-cell population. The down-regulation of effector T cell functions can include a selective inhibition of CD8⁺ T cell proliferation.

The invention further provides compositions of substantially pure hCG together with a pharmaceutically acceptable medium and formulations thereof. Such compositions can be used in a method of the invention to treat or inhibit a T cell mediated disease. For example, hCG can be administered as a solution or suspension together with a pharmaceutically acceptable medium. Such a pharmaceutically acceptable medium can be, for example, water, sodium phosphate buffer, phosphate buffered saline, normal saline or Ringer's solution or other physiologically buffered saline, or other solvent or vehicle such as a glycol, glycerol, oil or an injectable organic ester.

The heterodimeric hCG formulations of the invention include those applicable for parenteral administration such as subcutaneous, intraperitoneal, intramuscular, intravenous, intradermal, intracranial, intratracheal, and epidural administration. Heterodimeric hCG formulations of the invention also include those applicable for oral, rectal, ophthalmic (including intravitreal or intracameral), nasal, topical (including buccal and sublingual), intrauterine, or vaginal administration. The hCG formulations can be presented in unit dosage form and can be prepared by pharmaceutical techniques well known to those skilled in the art. Such techniques include the step of bringing into association the active ingredient and the pharmaceutical carrier(s) or excipient(s).

Formulations suitable for parenteral administration include aqueous and non-aqueous sterile injection solutions such as the pharmaceutically acceptable mediums described above. The solutions can additionally contain, for example, anti-oxidants, buffers, bacteriostats and solutes which render the formulation isotonic with the blood of the intended recipient. Other formulations include, for example, aqueous and non-aqueous sterile suspensions which can include suspending agents and thickening agents. The formulations can be presented in unit-dose or multi-dose containers, for example, sealed ampoules and vials, and can be stored in a lyophilized condition requiring, for example, the addition of a sterile liquid carrier prior to use. Extemporaneous injection solutions and suspensions can be prepared from sterile powders, granules and tablets of the kind previously described.

A pharmaceutically acceptable medium can additionally contain physiologically acceptable compounds that act, for example, to stabilize or increase receptor binding or antigen processing or presentation of hCG. Such physiologically acceptable compounds include, for example, carbohydrates such as glucose, sucrose or dextrans; antioxidants such as ascorbic acid or glutathione; chelating agents such as EDTA; divalent metal ions such as calcium or magnesium; low molecular weight proteins; lipids or liposomes; or other stabilizers or excipients. A heterodimeric hCG also can be formulated with a pharmaceutically acceptable medium such as a biodegradable polymer.

A heterodimeric hCG of the invention can be prepared or obtained by methods known in the art. For example, clinical grade hCG is readily available through a variety of commercial sources well known in the art. Additionally, hCG of the invention can be produced by biochemical purification. The choice of a particular method for purifying hCG will depend on the particular application of the protein by the user. For example, higher levels of purity may be desired for in vivo or therapeutic applications compared to in vitro confirmatory procedures. Alternatively, the α and β subunits of hCG can be chemically synthesized and allowed to self-assemble in vitro. The heterodimeric hCG can subsequently be isolated from the resultant products.

A hCG protein of the invention also can be recombinantly expressed by appropriate host cells, including bacteria, yeast, avian, insect and mammalian cells, using methods known in the art. Methods for recombinant expression and purification of polypeptides in various host organisms are described, for example, in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1989). Numerous methods for constructing, modifying, expression and purification are known to those skilled in the art. The choice of recombinant methods, expression and purification systems will be known by those skilled in the art and will depend on the user and the particular application for the hCG protein.

It is understood that modifications which do not substantially affect the activity of the various embodiments of this invention are also included within the definition of the invention provided herein. Accordingly, the following examples are intended to illustrate but not limit the present invention.

### EXAMPLE I

### Treatment of NOD Mice with hCG Prevents Autoimmune Diabetes

This Example shows that hCG treatment can prevent the onset of autoimmune diabetes.

Briefly, NOD mice and NOD.SCID mice were obtained from The Jackson Laboratory. NOD female mice of 3 or 10 weeks (wk) of age were used in the studies described herein. The animals were bred and maintained under specific pathogen-free conditions and provided with sterile food and water ad libitum at the Animal Resources Centre, Faculty of Medicine, University of Calgary. Female NOD mice were used throughout the studies. The use and care of the animals used in this study were approved by the Animal Care Committee, Faculty of Medicine, University of Calgary.

Mice were treated with clinical grade hCG (hCG) or recombinant hCG (rhCG) obtained from Sigma (St. Louis, Mo.). Purity of the hCG and rhCG was determined by 12% SDS-PAGE followed by protein staining using a silver staining kit (Bio-Rad, Hercules, Calif.). The pattern of protein bands in the clinical grade hCG preparation was shown to be the same as in the rhCG preparation, indicating that the clinical grade hCG did not contain other apparent protein contaminants compared with rhCG.

To determine the effect of hCG on the development of autoimmune diabetes in NOD mice, 50 IU/mouse of hCG in 200 μl PBS was injected intraperitoneally 5 times a wk from 3 wk of age to 15 wk of age and examined the development of diabetes. Control mice received 200 μl of PBS alone. The development of diabetes was monitored by urine measurements using Diastix (Bayer) every wk and positive glycosuria was confirmed by hyperglycemia (blood glucose level >16.7 mM).

Histology sections were prepared as follows. Briefly, pancreata were removed from hCG- or PBS-treated NOD mice sacrificed at 15 wk of age. The pancreata were fixed with 10% buffered formalin, embedded in paraffin, sectioned at 4.5 μM, and stained with hematoxylin and eosin (Yoon, et al. Science 284:1183-1187 ((1999); Chung, et al. J. Immunol. 165:2866-2876 (2000)). The degree of insulitis was scored as 4 follows: 0, intact islet; 1, <25% of the islet is infiltrated; 2, 25-50% of the islet is infiltrated; 3, 50-75% of the islet is infiltrated; 4, >75% of the islet is infiltrated.

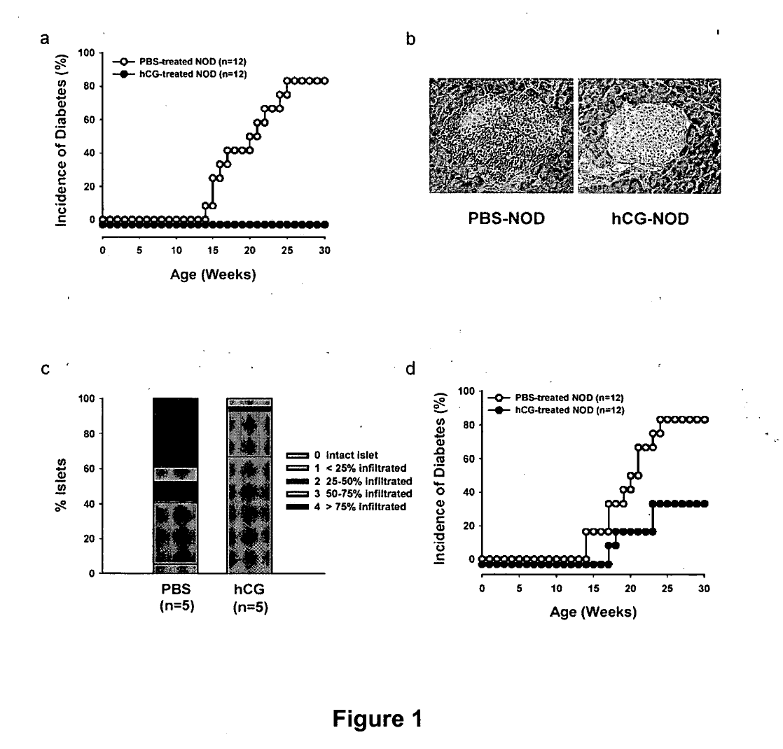

FIG. 1 shows the results hCG treatment of three (panel a) or ten (panel d) wk-old female NOD mice. The development of diabetes was monitored weekly. For comparison of incidence of diabetes, the Log-Rank test was used. Statistical scores showed a P<0.001 for mice treated with hCG from 3 wk of age (panel a) and P<0.01 for mice treated with hCG from 10 wk of age (panel d) as compared with PBS-treated mice. Data is representative of similar results from three independent experiments. Panels b and c show the results of five non-diabetic treated mice that were sacrificed at 15 wk of age and pancreatic sections were stained with hematoxylin and eosin. The degree of insulitis was examined in at least 20 islets/mouse (panel c) and photomicrographs were taken (panel b). Original magnification ×250.

The results of the above treatments shown in FIG. 1 indicate that none of the hCG-treated mice developed diabetes (0/12), whereas 83.3% (10/12) of the PBS-treated mice developed diabetes by 30 wk of age (FIG. 1a). When the animals were examined for insulitis at 15 wk of age, most of the islets from hCG-treated mice were found to be intact, whereas the majority of the islets from PBS-treated mice showed severe insulitis (FIGS. 1b, c).

To determine whether preventive effect also can be observed with treatment of hCG on the development of diabetes when administered to animals at an age when insulitis has substantially progressed, hCG was injected into 10 wk-old NOD female mice. The results shown in FIG. 1d indicate that the incidence of diabetes was significantly reduced in hCG-treated mice; 33% of hCG-treated NOD mice (4/12) developed diabetes, as compared with 83% of PBS-treated NOD mice (10/12). These results also indicate that treatment of NOD mice with hCG before the development of insulitis results in the complete prevention of autoimmune diabetes and that treatment after the development of insulitis results in a substantial decrease in the incidence of diabetes.

### EXAMPLE II

### Splenocytes from hCG-Treated NOD Mice Lose the Ability to Induce Diabetes in NOD.SCID Mice

This Example shows hCG treated splenocytes fail to induce diabetes when transferred to immunodeficient animals.

To determine whether splenocytes from hCG-treated NOD mice have the ability to transfer diabetes, splenocytes were isolated from hCG- or PBS-treated NOD mice at 15 wk of age and transferred to NOD.SCID mice. Recipient mice were examined for the development of diabetes.

Briefly, splenocytes isolated from hCG- or PBS-treated mice at 15 wk of age were transferred (i.v.) as described elsewhere (Jun supra; Utsugi, et al. Transplantation 57:1799-804 (1994)). An identical procedure also was used in studies described further below where mice were treated with rhCG. As a positive control for all adoptive transfer studies, splenocytes from acutely diabetic mice were injected into age-matched NOD.SCID mice (diabetic splenocytes). The development of diabetes in NOD.SCID recipients was monitored by measurement of urine glucose twice weekly and confirmed by measurements of blood glucose levels.

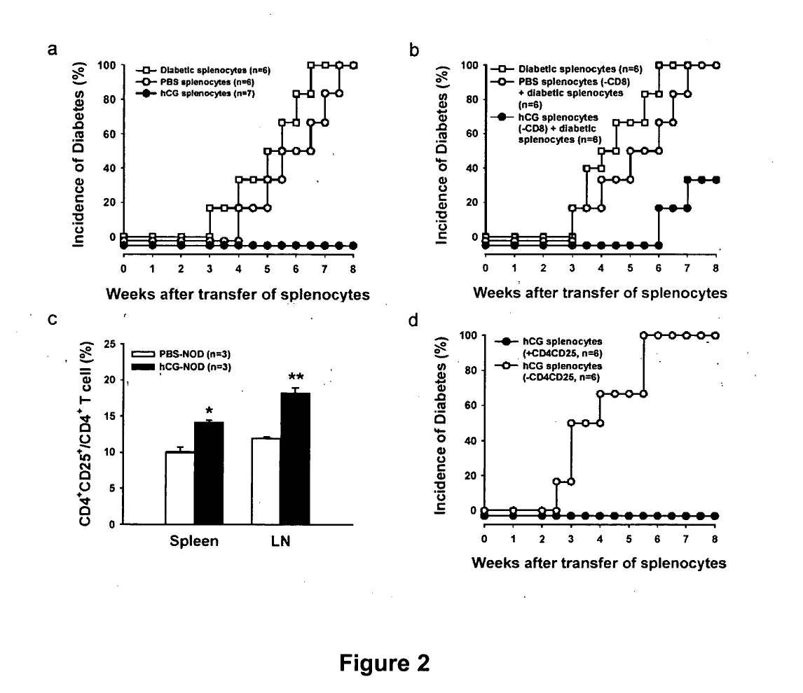

The results of these adoptive transfers are shown in FIG. 2(a) and (b). FIG. 2(a) shows splenocytes (1×10⁷ cells) isolated from PBS- or hCG-treated mice. The incidence of diabetes in recipients of hCG splenocytes was P<0.001 as compared with recipients of PBS splenocytes or diabetic splenocytes by Log-Rank test. The data shown in FIG. 2(a)-(d) are representative of similar results from three independent experiments.

The results show that 100% (6/6) of the recipients of splenocytes from PBS-treated mice developed diabetes by 8 wk after transfer. However, none (0/7) of the recipients of splenocytes from hCG-treated mice developed diabetes (FIG. 2a). This result indicates that splenocytes from hCG-treated NOD mice lose their ability to induce diabetes.

To determine whether splenocytes from hCG-treated NOD mice contain regulatory T cells that can inhibit the adoptive transfer of diabetes, CD8⁺ T cell-depleted splenocytes were co-transferred from hCG- or PBS-treated NOD mice along with splenocytes from acutely diabetic NOD mice into NOD.SCID mice and examined the development of diabetes. CD8⁺ T cells were depleted by negative selection. The CD8⁺ T cell-depleted splenocytes (1×10⁷ cells) were co-injected with splenocytes from acutely diabetic NOD mice (0.5×10⁷ cells, 2:1 ratio) into NOD.SCID mice and the development of diabetes was monitored. The incidence of diabetes was P<0.01 in recipients of “hCG splenocytes (−CD8)⁺ diabetic splenocytes” as compared with the incidence of diabetes in recipients of “PBS splenocytes (−CD8)⁺ diabetic splenocytes” or only “diabetic splenocytes”.

The results are shown in FIG. 2(b) and indicate that 100% (6/6) of NOD.SCID recipients of CD8⁺ T cell-depleted splenocytes from PBS-treated NOD mice along with splenocytes from diabetic mice developed diabetes within 8 wk after transfer. In contrast, only 33% (2/6) of NOD.SCID recipients of CD8⁺ T cell-depleted splenocytes from hCG-treated NOD mice along with splenocytes from diabetic mice developed diabetes (FIG. 2b). This result suggests that hCG-treatment may have induced a regulatory T cell population that can prevent the transfer of diabetes.

### EXAMPLE III

### Treatment of NOD Mice with hCG Selectively Increases the CD4⁺CD25⁺ Regulatory T Cell Population

This Example shows that hCG treatment selectively increases the CD4⁺CD25⁺ regulatory T cell population.

To determine whether treatment of NOD mice with hCG induces a regulatory T cell population, such as CD4⁺CD25⁺ T cells, splenocytes or pancreatic LN cells were isolated from hCG- or PBS-treated NOD mice at 9 wk of age and examined the CD4⁺CD25⁺ T cell population. The statistical significance of the differences between groups was analyzed by Student's t test. A level of P<0.05 was accepted as significant.

T cell proliferation and flow cytometric analysis (fluorescent activated cell sorting (FACS)) were performed as follows. Briefly, T cell proliferation assays employed splenocytes prepared from 15 wk-old female NOD mice treated with hCG or PBS. Splenocytes (1×10⁵ cells) were cultured for 96 h in the presence or absence of anti-CD3 Ab (0.5 μg/ml) or mouse insulinoma (MIN)₆N8a cell extract (25 μg/well) in 200 μl of complete RPMI medium containing 10% FBS and antibiotics in a 96-well microplate. For in vitro assay, splenocytes were prepared from 8 wk-old non-diabetic female NOD mice. Total splenocytes were stimulated with anti-CD3 Ab (0.5 μg/ml) in the presence or absence of hCG (100 IU/ml).

Proliferation of CD4⁺ and CD8⁺ T cells was assessed using splenocytes (1×10⁷ cells) obtained from 8 wk-old non-diabetic female NOD mice and labeled with 10 μl of anti-CD4 or anti-CD8 Ab-linked micromagnetic beads (Miltenyi Biotec, Auburn, Calif.) in 90 μl sorting buffer (PBS containing 0.5% BSA and 2 mM EDTA, pH 7.2) for 15 min at 4° C. CD4⁺- or CD8⁺-labeled splenocytes were washed with sorting buffer and loaded onto the selection column. The column was washed with sorting buffer, and CD4⁺ or CD8⁺ T cells were eluted. Purified CD4⁺ or CD8⁺ T cells (1×10⁵ cells) were stimulated with MIN6N8a cell extract (25 μg/well) for 96 h in the presence or absence of hCG (100 IU/ml). Irradiated, T cell-depleted splenocytes were added as APCs. The cells were pulsed with [³H]thymidine (1 μCi/well) for 16 h of incubation and then harvested. The incorporation of [³H]thymidine was measured by liquid scintillation counting (Young, et al. Science 284:1183-1187 (1999); Kawamura, et al. J. Immunol. 151:4362-4370 (1993)). A stimulation index (SI) was calculated by dividing the cpm from the stimulated group by the cpm from the unstimulated group.

FACS analysis employed splenocytes pancreatic LN cells were isolated from hCG, rhCG- or PBS-treated female NOD mice at 9 wk of age. The CD4⁺CD25⁺T cell population was examined by incubating cells (1×10⁶) with FITC-labeled anti-CD4 Ab and biotinylated anti-CD25 Ab (Pharmingen, Mississauga ON) for 30 min at 4° C. in staining buffer composed of PBS containing 1% FBS and 0.1% sodium azide. After washing, the cells were incubated with PE-labeled streptavidin (for CD25) for 30 min at 4° C. and washed twice with staining buffer and analyzed by FACS (Chung supra; Kawamura supra). CD4⁺ T cell and CD8⁺ T cell populations were examined using splenocytes stained with anti-CD4 or anti-CD8 Abs, respectively. NKT and CD4⁺CD62L⁺ regulatory cell populations were examined using splenocytes or LN cells that were double-stained with anti-CD3 and DX5 Abs or anti-CD4 and anti-CD62L Abs, respectively.

The results of this study are shown in FIG. 2(c) where splenocytes (spleen) and pancreatic LN cells (LN) isolated from PBS- or hCG-treated female NOD mice at 9 wk of age were double-stained with anti-CD4 and anti-CD25 Abs and analyzed by FACS. The statistical comparisons of cell populations also are shown where * corresponds to a P<0.01 and ** corresponds to a P<0.005 as compared with PBS treatment.

As shown in FIG. 2(c), the results indicate that CD4⁺CD25⁺ T cells in the spleen and pancreatic LN of hCG-treated NOD mice were increased by approximately 40% and 50%, respectively, as compared with PBS-treated NOD mice (P<0.01, P<0.005, FIG. 2c). NKT and CD4⁺CD62L⁺ T cell populations also were examined in splenocytes and pancreatic LN from hCG- or PBS-treated NOD mice. No significant difference in these regulatory T cell populations between hCG- and PBS treated mice was observed.

### EXAMPLE IV

### Depletion of CD4⁺CD25⁺ Regulatory T Cells Eliminates the Ability to Prevent Diabetes

This Example shows that hCG induced regulatory T cells play a preventative role in the onset or progression of diabetes.

To determine whether CD4⁺CD25⁺ T cells are responsible for the prevention of diabetes, CD4⁺CD25⁺ T cell populations were depleted from splenocytes of hCG-treated NOD mice and transferred the splenocytes to NOD.SCID recipients. Adoptive transfer of CD4⁺CD25⁺ T cells was performed as described previously. Briefly, splenocytes were isolated from hCG-treated NOD mice, and CD4⁺CD25⁺ T cells were depleted by negative selection using anti-CD4⁺ and anti-CD25⁺ Ab-linked micromagnetic beads (Miltenyi Biotec). The CD4⁺CD25⁺ T cell-depleted splenocytes or total splenocytes (1×10⁷ cells/recipient) were injected i.v. into 6 wk-old NOD.SCID mice.

The results of these studies are shown in FIG. 2(d) where splenocytes isolated from hCG-treated mice were divided into two parts and depletion was performed on one part. CD4⁺CD25⁺ T cell-depleted splenocytes (−CD4⁺CD25⁺) or total splenocytes (+CD4⁺CD25⁺). P<0.001 for the incidence of diabetes in recipients of hCG splenocytes containing CD4⁺CD25⁺ T cells as compared with recipients of hCG splenocytes not containing CD4⁺CD25⁺ T cells.

The results indicate that all the recipients of CD4⁺CD25⁺ T cell-depleted splenocytes developed diabetes, whereas none of the recipients of splenocytes containing CD4⁺CD25⁺ T cells developed diabetes (FIG. 2d). This result shows that CD4⁺CD25⁺ T cells are responsible for the prevention of diabetes in hCG-treated NOD mice.

### EXAMPLE V

### Treatment of NOD Mice with hCG Selectively Inhibits CD8⁺ T Cell Proliferation

This Example shows the hCG mediated inhibition of T cell effector cells.

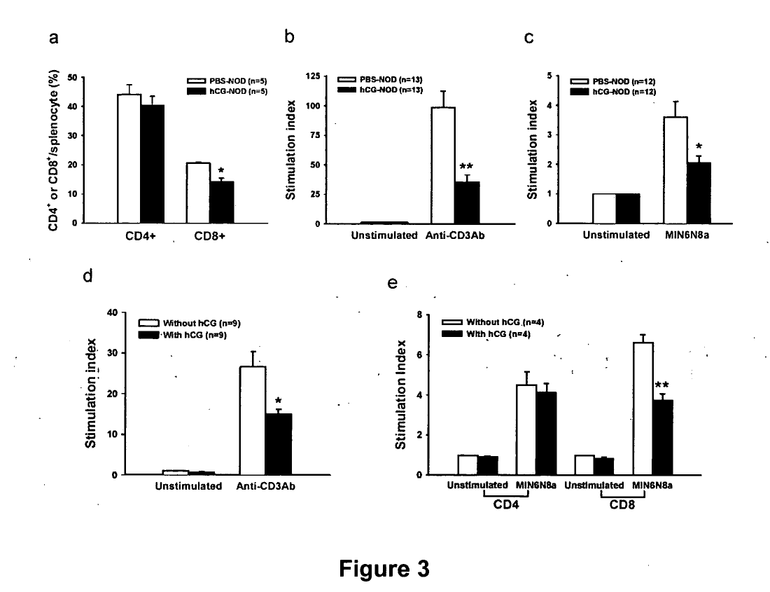

CD8⁺ T cells play an important role as final effectors in the destruction of pancreatic P cells, acting synergistically with Th1 CD4⁺ T cells and macrophages (Nagata, et al. J. Immunol. 152:2042-2050 (1994)). To assess whether hCG affects the generation of T cells in addition to the CD4⁺CD25⁺ subset, the proportion of the CD4⁺ and CD8⁺ T cell subpopulations was examined in the spleen of hCG-treated NOD mice at 15 wk of age by FACS analysis after staining with anti-CD4 and anti-CD8 Abs. FACS analysis and antibody staining were performed as described in the previous Examples and the results are shown in FIG. 3: Treatment of NOD mice with hCG resulted in a significant decrease in CD8⁺ T cells, but not CD4⁺ T cells in the spleen (FIG. 3a). For all results shown in FIG. 3, the symbol * corresponds to P<0.05 whereas ** corresponds to P<0.005 as compared with the PBS-treated group. Data are represented as means±SEM.

To assess whether hCG affects T cell proliferation, splenocytes were isolated from hCG or PBS-treated mice at 15 wk of age, activated with anti-CD3 Ab or β cell antigens (extracts of MIN6N8a cells), and examined the proliferative response.

FIGS. 3(b) and (c) show the stimulation index for splenocytes isolated from 15 wk-old, PBS- or hCG-treated female NOD mice stimulated with either (b) anti-CD3 Ab (0.5 μg/ml) or (c) MIN6N8a cell extract (25 μg/well). The results show that the proliferative response was significantly lower in hCG-treated NOD mice than PBS-treated NOD mice (FIGS. 3b, c). This result indicates that hCG can inhibit both antigen-specific (MIN6N8a) and non-specific (anti-CD3) T cell proliferation.

To determine whether hCG has a direct effect on T cell proliferation, splenocytes from 8 wk-old untreated NOD mice were isolated, activated with anti-CD3 Ab in vitro in the presence or absence of hCG, and the proliferative response examined. FIG. 3d shows that the in vitro T cell proliferative response was also significantly inhibited by hCG as compared with PBS. This result indicates that hCG directly inhibits T cell proliferation.

The demonstration that treatment of NOD mice with hCG resulted in a decrease in the number of CD8⁺ T cells, but not CD4⁺ T cells, in the spleen encouraged assessment of whether there was any difference in the capacity of CD8⁺ versus CD4⁺ T cells to proliferate. Splenic lymphocytes from 8 wk-old NOD mice were sorted into CD4⁺ and CD8⁺ T cell populations and stimulated in vitro with P cell antigens (MIN6N8a cell extract and APCs) in the presence or absence of hCG (100 IU/ml). The proliferative response of CD8⁺ T cells to MIN6N8a cell extract was inhibited to a significantly greater degree than the proliferative response of CD4⁺ T cells (FIG. 3e). This result indicates that CD8⁺ T cells are more sensitive to hCG-mediated inhibition of proliferation than CD4⁺ T cells.

### EXAMPLE VI

### Treatment of NOD Mice with hCG Down-Regulates the Th1 Immune Response

This Example shows hCG mediated down regulation of the Th1 immune response.

The previous Examples show that treatment of NOD mice with hCG up-regulated the CD4⁺CD25⁺ regulatory T cell population and down-regulated the CD8⁺ T cell population. To determine whether hCG also affects the Th1 immune response, splenocytes from hCG- or PBS-treated NOD mice at 15 wk of age were isolated, stimulated with anti-CD3 Ab, and the production of IFN-γ as a Th 1 cytokine and IL-4 as a Th2 cytokine was measured.

Cytokine production was determined by quantitative ELISA. Briefly, splenocytes were prepared from 15 wk-old female NOD mice treated with PBS, hCG, or 5 rhCG. Splenocytes (1×10⁶ cells) were stimulated with anti-CD3 Ab (0.5 μg/ml) for 24 or 48 h in 1 ml of complete RPMI medium in a 24-well plate. For in vitro assay, splenocytes were prepared from 8 wk-old female NOD mice and stimulated with anti-CD3 Ab in the presence or absence of hCG (100 IU/ml). For investigation of macrophage function, splenocytes (2×10⁶ cells) isolated from PBS-, hCG-, or rhCG-treated NOD mice were attached on 6-well plates for 1 h at 37° C. in a CO₂ incubator and washed with PBS to remove unattached cells. Attached macrophages were stimulated with LPS (10 ng/ml) for 24 h. For in vitro assay, 8 wk-old female NOD mice were injected with 2 ml 4% thioglycolate i.p.; 4 days later, peritoneal macrophages were obtained from the cavity with 5 ml complete RPMI medium containing 2% FBS. The purity of the macrophages was over 90% as determined by FACS with anti-CD11b Ab. Macrophages (1×10⁶ cells) were attached on 12-well plates and stimulated with LPS (10 ng/ml) in the presence or absence of hCG (100 IU/ml) for 24 h. The supernatant was collected and cytokine release was measured using a Quantikine ELISA kit (R & D Systems, Minneapolis Minn.), according to the manufacturer's protocol. Cell pellets were saved for RT-PCR analysis of cytokine mRNA expression.

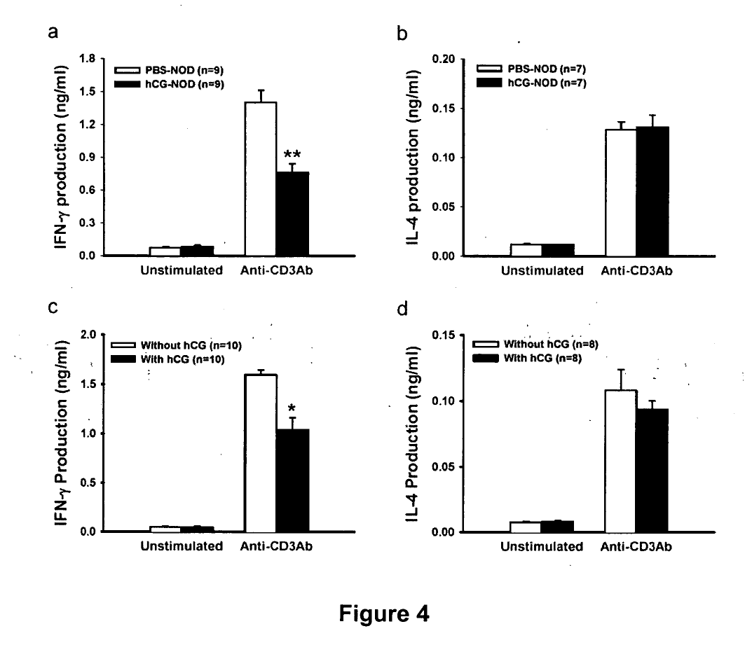

The results of this study are shown in FIG. 4. Panel (a) shows the production of IFN-γ whereas panel (b) shows the production IL-4. The results indicate that hCG treatment resulted in the significant inhibition of IFN-γ production (FIG. 4a), but did not affect IL-4 production (FIG. 4b).

To determine the direct effect of hCG on the production of IFN-γ and IL-4 in vitro, splenocytes were isolated from 8 wk-old, untreated female NOD mice, stimulated with anti-CD3 Ab for 24 h for IFN-γ or 48 h for IL-4 in the presence or absence of hCG (100 IU/ml) and the production of IFN-γ and IL-4 was determined by ELISA. The results are shown in Figure (c) and (d), respectively. The symbol * corresponds to P<0.01 whereas ** corresponds to P<0.005 as compared with the PBS-treated group. Data are represented as the means±SEM. Similar to the in vivo results, hCG treatment significantly inhibited IFN-γ production, but did not affect IL-4 production (FIGS. 4c, d). These results indicate that hCG treatment can down-regulate the Th1 immune response.

### EXAMPLE VII

### Treatment of NOD Mice with hCG Inhibits Macrophage Activation

This Example shows hCG mediated inhibition of macrophage activation.

Activated macrophages have been shown to be primary contributors to activation of T cells that destroy pancreatic β cells and produce autoimmune diabetes in NOD mice (Jun, et al. J. Exp. Med. 189:347-358 (1999)). Depletion of macrophages in young NOD mice prevents insulitis and diabetes by decreasing the Th1 immune response and reducing the expression of macrophage-derived cytokines (Jun supra). To determine whether hCG affects macrophage activation, TNF-α production and IL-1β and iNOS gene expression was examined in macrophages from hCG-treated NOD mice.

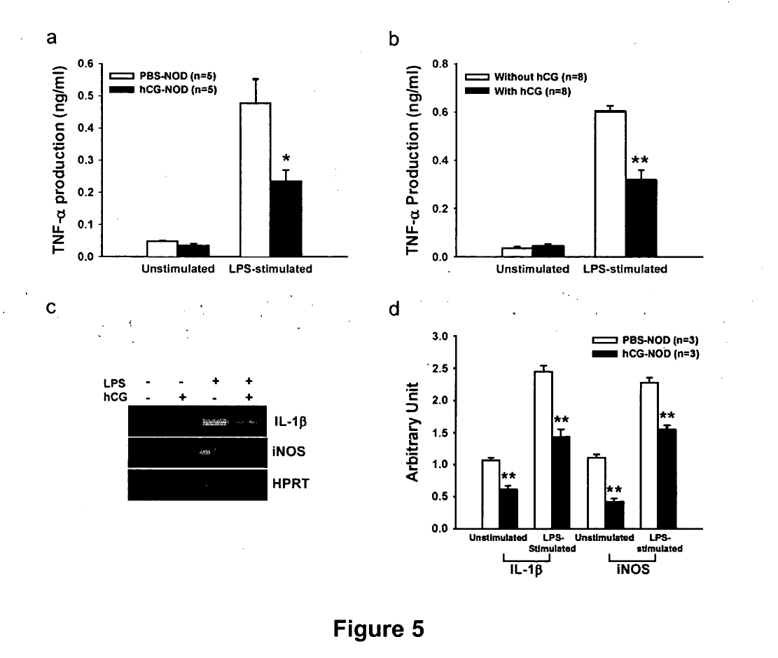

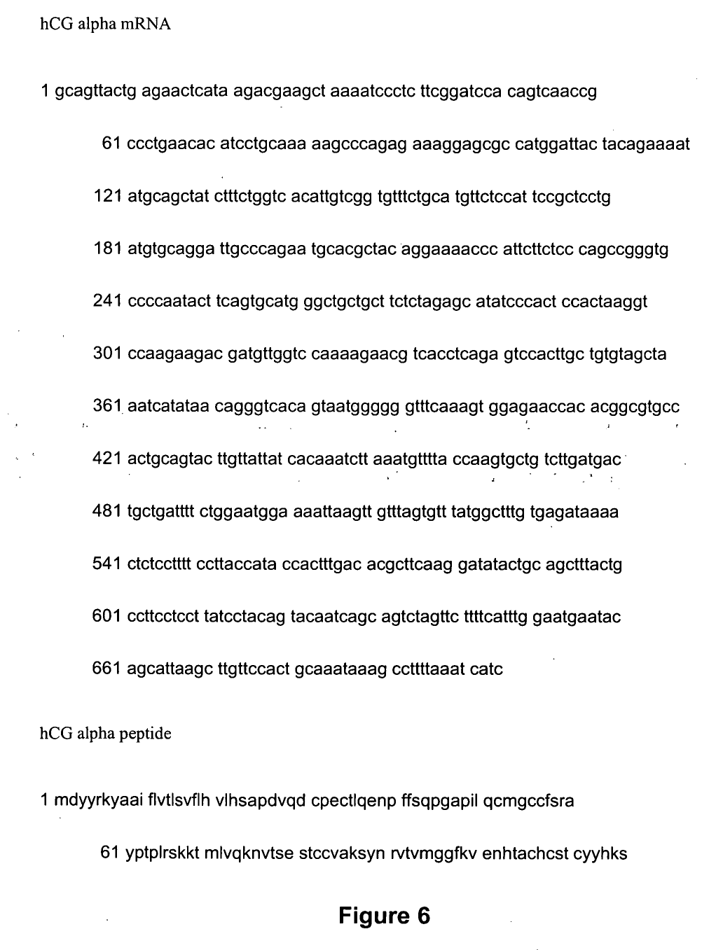

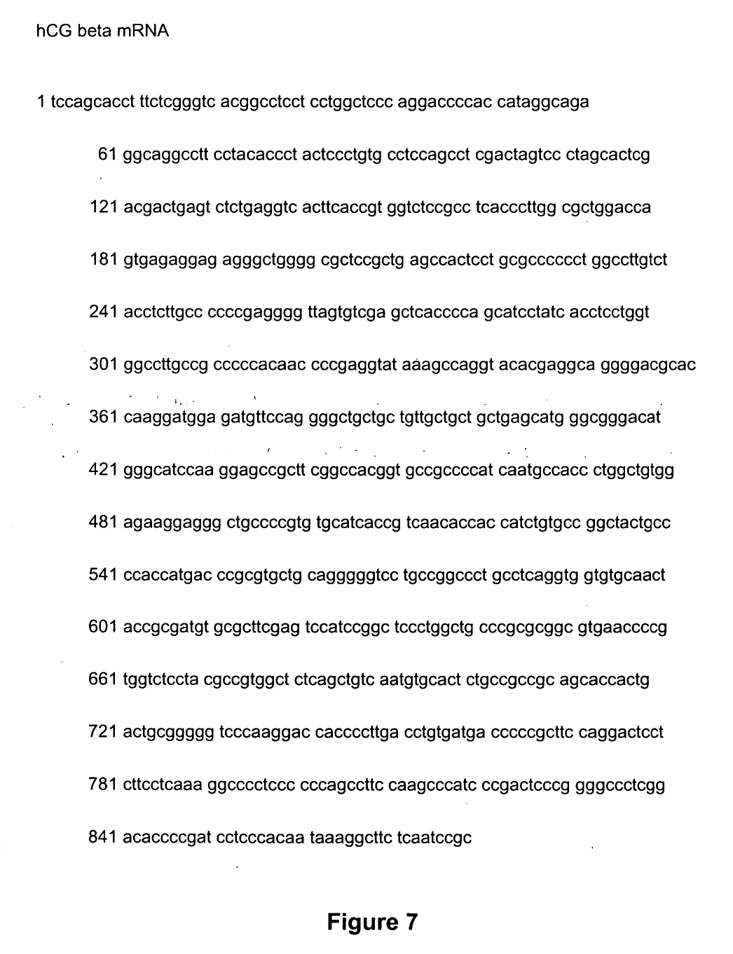

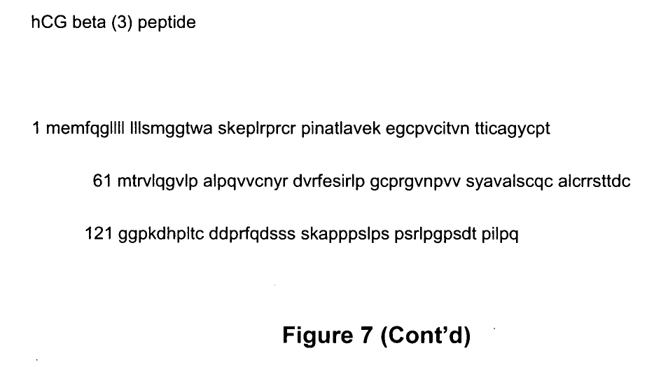

Macrophages were isolated from splenocytes of PBS- or hCG-treated 15 wk-old female NOD mice and stimulated in vitro with LPS (10 ng/ml) for 24 h. TNF-α production was measured by ELISA. FIG. 5(a) shows that TNF-α production was significantly decreased in the hCG-treated group as compared with the PBS-treated group.

To determine the effect of hCG on TNF-α production in vitro, peritoneal macrophages isolated from 8 wk-old untreated NOD mice were stimulated with LPS for 24 h in the presence or absence of hCG (100 IU/ml). The results are shown in FIG. 5(b) and indicate that hCG treatment significantly inhibited TNF-α production in vitro.

The expression level of IL-1β and iNOS also was measured in isolated macrophages from hCG- or PBS-treated NOD mice by RT-PCR.

Briefly, total RNA was extracted from peritoneal macrophages, obtained from 8 wk-old untreated NOD mice that were stimulated in vitro for 24 h with LPS in the presence or absence of hCG.using Trizol (Gibco BRL, Gaithersburg, Md.) according to the manufacturer's protocol. Two μg of total RNA was used to synthesize cDNA using Superscript II reverse transcriptase (Gibco BRL) and oligo(dT) 12-18. PCR was performed with specific primers for various cytokine genes, as previously described (Jun supra). The upstream and downstream primers for IL-1β were: sense, 5′-GAATGACCTGTTCTTTGAAGTT-3′ (SEQ ID NO:5); antisense, 5′-TTTTGTTGTTCATCTCGGAGCC-3′ (SEQ ID NO:6) and for iNOS were: sense, 5′-CCTTCCGAAGTTTCTGGCAGCAGC-3′ (SEQ ID NO:7); antisense, 5′-GGCTGTCAGAGCCTCGTGGCTTTGG-3′ (SEQ ID NO:8). Hypoxanthine phosphoribosyl transferase (HPRT) was used as an internal standard. The primers for HPRT were: sense, 5′-GTAATGATCAGTCAACGGGGGAC-3′ (SEQ ID NO:9); antisense, 5′-CCAGCAAGCTTGCAACCTTAACCA-3′ (SEQ ID NO:10). The PCR condition was optimized for each set of primers. PCR was performed using different numbers of cycles to ensure that amplification occurred in a linear range. The PCR mixture (50 μl) contained 0.2 mM of each deoxynucleotide triphosphate, 1 μM of each specific primer, 1.5 mM MgCl2, 50 mM KCl, 10 mM Tris-Ci, pH 9.0, and 2.5 U of Taq polymerase (Gibco BRL). After amplification, the products were subjected to electrophoresis on a 1% agarose gel and detected by ethidium bromide staining.

The results of these expression analysis are shown in FIG. 5(c). FIG. 5(d) shows the results of IL-1β and iNOS bands that were normalized with HPRT and the relative amount of mRNA in each group was calculated. Data are the means±SEM. The symbol * in FIG. 5 represents P<0.05 where as ** represents P<0.005 as compared with the PBS-treated group.

Treatment with hCG suppressed both IL-1β and iNOS gene expression (FIGS. 5c, d) and indicate that treatment of NOD mice with hCG results in the inhibition of macrophage activation.

### EXAMPLE VIII

### Effect of Recombinant hCG on the Control of Autoimmune Diabetes is Comparable to that of Purified hCG in NOD Mice

This Example shows comparative effects between recombinant hCG (rhCG) and hCG on the prevention of diabetes in animal models.

To determine whether recombinant hCG (rhCG) has a similar effect on the control of autoimmune diabetes as purified hCG, 3 wk-old female NOD mice were treated with rhCG or hCG and examined the incidence of diabetes, the induction of CD4⁺CD25⁺ regulatory T cells, and the expression of cytokines. The results are shown in Table 1 and indicate that rhCG prevented the development of diabetes in NOD mice nearly to the same degree as hCG. Moreover, neither NOD.SCID mice that received splenocytes from hCG- nor rhCG-treated mice developed diabetes. The CD4⁺CD25⁺ regulatory T cell population was significantly increased in both hCG- and rhCG-treated NOD mice as compared with PBS-treated mice, but to a slightly less degree in rhCG-treated mice. Both hCG and rhCG inhibited the production of IFN-γ and TNF-α, but neither inhibited the production of IL-4. These results with rhCG further indicate that hCG or a fragment of hCG, but not a contaminant in the hCG preparation, is responsible for the prevention of autoimmune diabetes in NOD mice.

TABLE 1
Comparison of recombinant hCG (rhCG) with clinical grade hCG
(hCG) on the control of autoimmune diabetes in NOD mice.

| | PBS | hCG | rhCG |
|---|---|---|---|
| Incidence of | 10/12 (83.3%) | 0/12 (0%)^{H} | 1/6 (167%)^{E} |
| diabetes^{A} | | | |
| Incidence of | 6/6 (100%) | 0/6 (0%)^{H} | 0/6 (0%)^{H} |
| diabetes by adoptive | | | |
| transfer^{B} | | | |
| Expression of | | | |
| cytokines (ng/ml)^{C} | | | |
| IFN-γ (n = 9) | 1.400 ± 0.115 | 0.764 ± 0.077^{G} | 0.790 ± 0.0921^{F} |
| IL-4 (n = 7) | 0.129 ± 0.011 | 0.131 ± 0.012 | 0.133 ± 0.013 |
| TNF-α (n = 5) | 0.479 ± 0.076 | 0.236 ± 0.033^{E} | 0.287 ± 0.030^{E} |
| Proportion of | | | |
| CD4⁺CD25⁺ | | | |
| T cells^{D} | | | |
| Spleen (n = 3) | 10.0 ± 0.7% | 14.1 ± 0.5%^{F} | 12.9 ± 0.4%^{E} |
| Pancreatic LN | 11.9 ± 0.3% | 18.2 ± 1.3%^{G} | 15.2 ± 0.8%^{G} |
| (n = 3) | | | |

^{A}3 wk-old female NOD mice were injected i.p. with 50 IU hCG, 50 IU rhCG, or PBS five times a wk for 12 wk. The cumulative incidence of diabetes at 30 wk of age was determined.
^{B}Splenocytes (1 × 10⁷ cells) isolated from 15 wk-old female NOD mice treated with hCG, rhCG, or PBS for 12 wk were transferred into 6 wk-old NOD.SCID mice. The cumulative incidence of diabetes in the NOD.SCID recipients was determined at 8 wk after transfer of splenocytes.
^{C}The production of IFN-γ and IL-4 in splenocytes and TNF-α in macrophages isolated from 15 wk-old female NOD mice treated with hCG, rhCG, or PBS for 12 wk was measured by 2829 ELISA.
^{D}Splenocytes and pancreatic LN cells isolated from hCG-, rhCG-, or PBS-treated female NOD mice (9 wk of age) were double-stained with anti-CD4 and anti-CD25 Abs and analyzed by FACS.
Significance when compared with the PBS control:
^{E}P ≦ 0.05,
^{F}P ≦ 0.01,
^{G}P ≦ 0.005,
^{H}P ≦ 0.001.
Data are means ± SEM.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A method of treating a T cell mediated disease, comprising administering an effective amount of hCG to an asymptomatic subject over a period of time sufficient to restore persistent immune balance between regulatory and effector T cells, wherein restoring said persistent immune balance results in a reduction in the severity of said T cell mediated disease.

2. The method of claim 1, wherein said effective amount is administered prior to or concurrent with the occurrence of autoimmune dysfunction.

3. The method of claim 2, wherein said reduction in the severity of said T cell mediated disease comprises prevention of disease onset.

4. The method of claim 1, wherein said effective amount is administered subsequent to the occurrence of autoimmune dysfunction.

5. The method of claim 4, wherein said reduction in the severity of said T cell mediated disease comprises reducing the progression or symptoms of said T cell mediated disease.

6. The method of claim 1, wherein said T cell mediated disease comprises type 1 diabetes.

7. The method of claim 1, wherein said T cell mediated disease comprises insulitis.

8. The method of claim 1, wherein said T cell mediated disease is selected from the group consisting of Graves' disease, rheumatoid arthritis (RA), multiple sclerosis (MS), systemic lupus erythematosus, myasthenia gravis and pemphigus vulgaris.

9. The method of claim 1, wherein restoring said persistent immune balance between regulatory and effector T cells comprises an up-regulation of regulatory T cell function, a down-regulation of effector T cell function or both the up-regulation of regulatory T cell functions and the down-regulation of effector T cell functions.

10. The method of claim 9, wherein said up-regulation of regulatory T-cell function comprises an increase in CD4⁺CD25⁺ regulatory T cell population.

11. The method of claim 9, wherein said down-regulation of effector T cell function comprises a selective inhibition of CD8⁺ T cell proliferation.

12. The method of claim 1, wherein said effective amount of hCG comprises between about 5-500 IU/kg body weight, particularly between about 10-250 IU/kg body weight, and more particularly, between about 15-200 IU/kg body weight.

13. A method of preventing T cell mediated disease in a pre-diseased subject, comprising administering an effective amount of hCG to a subject at risk of developing a T cell mediated disease for sufficient duration to confer persistent immune balance between regulatory and effector T cells functions.

14. The method of claim 13, wherein said T cell mediated disease comprises type 1 diabetes.

15. The method of claim 13, wherein said T cell mediated disease comprises insulitis.

16. The method of claim 13, wherein said T cell mediated disease is selected from the group consisting of Graves' disease, rheumatoid arthritis (RA), multiple sclerosis (MS), systemic lupus erythematosus, myasthenia gravis and pemphigus vulgaris.

17. The method of claim 13, wherein restoring said persistent immune balance between regulatory and effector T cells comprises an up-regulation of regulatory T cell function, a down-regulation of effector T cell function or both the up-regulation of regulatory T cell functions and the down-regulation of effector T cell functions.

18. The method of claim 17, wherein said up-regulation of regulatory T-cell function comprises an increase in CD4⁺CD25⁺ regulatory T cell population.

19. The method of claim 17, wherein said down-regulation of effector T cell function comprises a selective inhibition of CD8⁺ T cell proliferation.

20. The method of claim 13, wherein said effective amount of hCG comprises between about 5-500 IU/kg body weight, particularly between about 10-250 IU/kg body weight, and more particularly, between about 15-200 IU/kg body weight.

21. A pharmaceutical composition, comprising an effective amount of substantially purified hCG in a pharmaceutically acceptable medium and a formulations thereof suitable for administration to a subject.
